(19) United States
(12) Patent Application Publication
(10) Pub. No.: US 2006/0241435 A1
(43) Pub. Date: Oct. 26, 2006

(54) Ultrasound diagnostic apparatus

(75) Inventors: Yumiko Koga, Tokyo (JP); Takekazu Osada, Tokyo (JP); Kazuhiko Hayakawa, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/401,159

(22) Filed: Apr. 10, 2006

(30) Foreign Application Priority Data
Apr. 11, 2005 (JP) 2005-113034

(51) Int. Cl. A61B 8/00

(52) U.S. Cl. 600/437

(57) ABSTRACT

The present invention aims to provide an ultrasound diagnostic apparatus that can move a display screen for an ultrasound image to a position from which the display screen is easy to see. An image display device is supported by an image display device supporting mechanism so as to be horizontally movable and vertically movable and so as to be capable of changing the tilt angle of the display screen of the image display device. A neck member is brought into contact with a stopper member and a lower end of a rod is inserted into a cutout, whereby the image display device is locked at the tilt angle in which the display screen faces downward. A stopper member and a guide member are brought into contact with each other, thereby restricting the downward movement of the image display device.

### CROSS REFERENCE TO RELATED APPLICATIONS

This application claims benefit of Japanese Application No. 2005-113034 filed Apr. 11, 2005.

### BACKGROUND OF THE INVENTION

The present invention relates to an ultrasound diagnostic apparatus, and more particularly to an ultrasound diagnostic apparatus that can move a display screen of an ultrasound image to a position from which the image is easy to be seen.

There has been known an ultrasound diagnostic apparatus wherein a liquid crystal display device is mounted to an ultrasound diagnostic apparatus body by a hinge (e.g., see Patent Reference 1).

[Patent Reference] Japanese Unexamined Patent Application No. 2002-272739

In the above-mentioned conventional ultrasound diagnostic apparatus, the liquid crystal display device is raised to display an ultrasound image when it is used, while the liquid crystal display device is thrown down when not used, so that it can be easy to be carried.

However, it has a problem that the liquid crystal display device cannot be moved in the horizontal direction or vertical direction to move the display screen to a position from which the display screen is easy to be seen.

### SUMMARY OF THE INVENTION

Therefore, an object of the present invention is to provide an ultrasound diagnostic apparatus that can move a display screen for an ultrasound diagnostic image to a position from which the display screen is easy to be seen.

In the first aspect, the invention provides an ultrasound diagnostic apparatus comprising: an ultrasound diagnostic apparatus body performing a control of an ultrasound probe or a formation of an ultrasound image; an image display device that displays the ultrasound image; and an image display device supporting mechanism that supports the image display device to the ultrasound diagnostic apparatus body, wherein the image display device supporting mechanism supports the image display device so as to be horizontally movable and vertically movable and so as to be capable of changing the tilt angle of a display screen of the image display device, as well as is capable of supporting the image display device in a state where the image display device is thrown down with the display screen facing downward, and the image display device supporting mechanism comprises tilt lock means for locking the tilt angle of the display screen in the state where the image display device is thrown down with the display screen facing downward, and movable range restricting means for restricting a horizontally movable range and a vertically movable range in accordance with the horizontal position and the vertical position of the image display device.

According to the ultrasound diagnostic apparatus in the first aspect, the image display device is supported, via the image display device supporting mechanism, to the ultrasound diagnostic apparatus body so as to be horizontally movable and vertically movable and so as to be capable of changing the tilt angle of the display screen of the image display device, thereby being capable of moving the display screen for the ultrasound diagnostic image to a position and posture by which the display screen is easy to be seen. Further, the image display device is thrown down and the tilt angle of the display screen is locked, when not used, whereby it becomes easy to be carried. Moreover, the horizontally movable range and the vertically movable range are restricted according to the horizontal position and the vertical position of the image display device, thereby being capable of avoiding the interference between the ultrasound diagnostic apparatus body and the image display device.

In the second aspect, the present invention provides an ultrasound diagnostic apparatus comprising: an ultrasound diagnostic apparatus body performing a control of an ultrasound probe or a formation of an ultrasound image; an image display device that displays the ultrasound image; and an image display device supporting mechanism that supports the image display device to the ultrasound diagnostic apparatus body, wherein the image display device supporting mechanism supports the image display device so as to be horizontally movable and vertically movable and so as to be capable of changing the tilt angle of a display screen of the image display device, as well as is capable of supporting the image display device in a state where the image display device is thrown down with the display screen facing downward.

According to the ultrasound diagnostic apparatus in the second aspect, the image display device is supported, via the image display device supporting mechanism, to the ultrasound diagnostic apparatus body so as to be horizontally movable and vertically movable and so as to be capable of changing the tilt angle of the display screen of the image display device, thereby being capable of moving the display screen for the ultrasound diagnostic image to a position and posture by which the display screen is easy to be seen. Further, the image display device can be thrown down when not used, whereby it becomes easy to be carried.

In the third aspect, the present invention provides, in the ultrasound diagnostic apparatus according to the first and second aspects, an ultrasound diagnostic apparatus comprising tilt lock means for locking the tilt angle of the display screen in the state where the image display device is thrown down with the display screen facing downward.

According to the ultrasound diagnostic apparatus in the third aspect, the tilt angle of the display screen is locked with the image display device thrown down, when not used, whereby it becomes easy to be carried.

In the fourth aspect, the present invention provides, in the ultrasound diagnostic apparatus according to any one of the first to third aspects, an ultrasound diagnostic apparatus wherein the image display device supporting mechanism has horizontal movement lock means for locking the horizontal position of the image display device when the tilt angle is locked by the tilt lock means.

According to the ultrasound diagnostic apparatus in the fourth aspect, the horizontal movement of the image display device can also be locked when the tilt angle of the display screen is locked with the image display device thrown down, whereby it becomes easy to be carried.

In the fifth aspect, the present invention provides, in the ultrasound diagnostic apparatus according to the fourth aspect, an ultrasound diagnostic apparatus wherein the horizontal movement lock means returns the image display device to the horizontal position when the lock of the tilt angle by the tilt lock means is released and the image display device is raised such that the display screen faces frontward.

According to the ultrasound diagnostic apparatus in the fifth aspect, when the liquid crystal display device is raised when used, the lock of the horizontal movement of the image display device is released with this, whereby the image display device can be horizontally moved to a position from which the image display device is easy to be seen.

In the sixth aspect, the present invention provides, in the ultrasound diagnostic apparatus according to any one of the first to fifth aspects, an ultrasound diagnostic apparatus wherein the lock of the tilt angle by the tilt lock means is possible only in the state where the image display device is at a specific horizontal position.

According to the ultrasound diagnostic apparatus in the sixth aspect, the tilt angle can be locked when the image display device is located at the specific, easy-to-carry, horizontal position.

In the seventh aspect, the present invention provides, in the ultrasound diagnostic apparatus according to any one of the first to sixth aspects, an ultrasound diagnostic apparatus wherein the image display device supporting mechanism has a rotary section that is rotatable in the horizontal direction with respect to the ultrasound diagnostic apparatus body, an arm section having a first end side supported by the rotary section and a second end side that can be pivoted in the upward and downward directions with the first end side as a support and a neck section having one end supported by the second end side of the arm section and the other end side that can be pivoted in the upward and downward directions with the one end side as a support, the image display device being supported to the other end side.

According to the ultrasound diagnostic apparatus in the seventh aspect, the horizontal movement is possible at the rotary section, the vertical movement is possible at the arm section, and the change of the tilt angle of the display screen is possible at the neck section.

In the eighth aspect, the present invention provides, in the ultrasound diagnostic apparatus according to the seventh aspect, an ultrasound diagnostic apparatus wherein the tilt lock means is means for locking the pivot movement of the neck section.

According to the ultrasound diagnostic apparatus in the eighth aspect, the lock of the tilt angle of the display screen is possible at the neck section.

In the ninth aspect, the present invention provides, in the ultrasound diagnostic apparatus according to the seventh or eighth aspect, an ultrasound diagnostic apparatus wherein the horizontal movement lock means is means for locking the rotation of the rotary section.

According to the ultrasound diagnostic apparatus in the ninth aspect, the lock of the horizontal movement of the image display device is possible at the rotary section.

In the tenth aspect, the present invention provides an ultrasound diagnostic apparatus comprising: an ultrasound diagnostic apparatus body performing a control of an ultrasound probe or a formation of an ultrasound image; an image display device that displays the ultrasound image; and an image display device supporting mechanism that supports the image display device to the ultrasound diagnostic apparatus body, wherein the image display device supporting mechanism supports the image display device so as to be horizontally movable and vertically movable and has movable range restricting means for restricting a horizontally movable range and vertically movable range in accordance with the horizontal position and the vertical position of the image display device.

According to the ultrasound diagnostic apparatus in the tenth aspect, the image display device is supported, via the image display device supporting mechanism, to the ultrasound diagnostic apparatus body so as to be horizontally movable and vertically movable, thereby being capable of moving the display screen for the ultrasound diagnostic image to a position and posture by which the display screen is easy to be seen. Moreover, the horizontally movable range and the vertically movable range are restricted according to the horizontal position and the vertical position of the image display device, thereby being capable of avoiding the interference between the ultrasound diagnostic apparatus body and the image display device.

In the eleventh aspect, the present invention provides, in the ultrasound diagnostic apparatus according to the first or tenth aspect, an ultrasound diagnostic apparatus wherein the movable range restricting means restricts the vertically movable range such that, at the position where the downmost point of the image display device is positioned above the topmost point of the ultrasound diagnostic apparatus body and the rightmost point of the image display device is positioned between the leftmost point and the rightmost point of the ultrasound diagnostic apparatus body, the downmost point of the image display device does not lower from the topmost point of the ultrasound diagnostic apparatus body, the movable range restricting means restricts the vertically movable range such that, at the position where the downmost point of the image display device is positioned above the topmost point of the ultrasound diagnostic apparatus body and the leftmost point of the image display device is positioned between the leftmost point and the rightmost point of the ultrasound diagnostic apparatus body, the downmost point of the image display device does not lower from the topmost point of the ultrasound diagnostic apparatus body, the movable range restricting means restricts the horizontally movable range such that, at the vertical position where the rightmost point of the image display device is positioned at the left side from the leftmost point of the ultrasound diagnostic apparatus body and the downmost point of the image display device is below the topmost point of the ultrasound diagnostic apparatus body, the rightmost point of the image display device does not move to the right from the leftmost point of the ultrasound diagnostic apparatus body, and the movable range restricting means restricts the horizontally movable range such that, at the vertical position where the leftmost point of the image display device is positioned at the right side from the rightmost point of the ultrasound diagnostic apparatus body and the downmost point of the image display device is below the topmost point of the ultrasound diagnostic apparatus body, the leftmost point of the image display device does not move to the left from the rightmost point of the ultrasound diagnostic apparatus body.

According to the ultrasound diagnostic apparatus in the eleventh aspect, the horizontally movable range and the vertically movable range can be restricted according to the horizontal position and the vertical position of the image display device.

In the twelfth aspect, the present invention provides, in the ultrasound diagnostic apparatus according to the tenth or eleventh aspect, an ultrasound diagnostic apparatus wherein the image display device supporting mechanism has a rotary section that is rotatable in the horizontal direction with respect to the ultrasound diagnostic apparatus body, and an arm section that has the first end side supported to the rotary section, and has the second end side that supports the image display device and can be pivoted in the upward and downward directions with the first end side as a support, the image display device being supported to the second end side.

According to the ultrasound diagnostic apparatus in the twelfth aspect, the horizontal movement is possible at the rotary section and the vertical movement is possible at the arm section.

In the thirteenth aspect, the present invention provides, in the ultrasound diagnostic apparatus according to the twelfth aspect, an ultrasound diagnostic apparatus comprising a neck section having one end supported by the second end side of the arm section and the other end side that can be pivoted in the upward and downward directions with the one end side as a support, the image display device being supported to the other end side.

According to the ultrasound diagnostic apparatus in the thirteenth aspect, the change of the tilt angle of the display screen is possible at the neck section.

In the fourteenth aspect, the present invention provides, in the ultrasound diagnostic apparatus according to the twelfth or thirteenth aspect, an ultrasound diagnostic apparatus wherein the movable range restricting means has a guide member that is located at a fixed position with respect to the ultrasound diagnostic apparatus body and a stopper member that moves with the arm member, wherein the stopper member is brought into contact with a part of the guide member, whereby the movable ranges of the rotary section and the arm section are restricted.

According to the ultrasound diagnostic apparatus in the fourteenth aspect, the stopper member is brought into contact with a part of the guide member, whereby the horizontally movable range and the vertically movable range can be restricted according to the horizontal position and the vertical position of the image display device for avoiding the interference between the ultrasound diagnostic apparatus body and the image display device.

In the fifteenth aspect, the present invention provides, in the ultrasound diagnostic apparatus according to any one of the first to fourteenth aspects, an ultrasound diagnostic apparatus wherein the image display device supporting mechanism maintains the tilt angle of the display screen of the image display device fixed even if the vertical position of the image display device is changed.

According to the ultrasound diagnostic apparatus in the fifteenth aspect, it is unnecessary to readjust the tilt angle of the display screen of the image display device every time the vertical position of the image display device is changed.

In the sixteenth aspect, the present invention provides, in the ultrasound diagnostic apparatus according to any one of the first to fifteenth aspects, an ultrasound diagnostic apparatus wherein the image display device is a flat panel type image display device.

According to the ultrasound diagnostic apparatus in the sixteenth aspect, it becomes possible to reduce the size of the image display device.

In the seventeenth aspect, the present invention provides, in the ultrasound diagnostic apparatus according to the sixteenth aspect, an ultrasound diagnostic apparatus wherein the flat panel type image display device is a liquid crystal display device.

According to the ultrasound diagnostic apparatus in the seventeenth aspect, it becomes possible to reduce the size of the image display device.

The ultrasound diagnostic apparatus of the present invention makes it possible to move the display screen for an ultrasound image to a position from which the display screen is easy to be seen according to the condition of an operator of the ultrasound diagnostic apparatus.

The ultrasound diagnostic apparatus according to the present invention can be utilized for imaging an ultrasound image.

Further objects and advantages of the present invention will be apparent from the following description of the preferred embodiments of the invention as illustrated in the accompanying drawings.

### BRIEF DESCRIPTION OF THE DRAWINGS

FIG. 1 is a partially cutaway left side view showing a using state of an ultrasound diagnostic apparatus according to an embodiment 1.

FIG. 2 is a top view of an essential section showing a horizontal movement restricting configuration in FIG. 1.

FIG. 3 is a partially cutaway left side view showing a carrying state of the ultrasound diagnostic apparatus according to the embodiment 1.

FIG. 4 is a top view of an essential section showing a horizontal movement restricting configuration in FIG. 3.

FIG. 5 is a partially cutaway left side view showing state where an image display device is raised in the ultrasound diagnostic apparatus according to the embodiment 1.

FIG. 6 is a partially cutaway left side view showing state where the image display device is lowered in the ultrasound diagnostic apparatus according to the embodiment 1.

FIG. 7 is a front view of an essential section showing a vertical movement restricting configuration in FIG. 6.

FIG. 8 is a partially cutaway left side view showing a carrying state of the ultrasound diagnostic apparatus according to an embodiment 2.

FIG. 9 is a partially cutaway left side view showing a carrying state of the ultrasound diagnostic apparatus according to an embodiment 3.

FIG. 10 is a front view of an essential section showing a vertical movement restricting configuration in FIG. 9.

### DETAILED DESCRIPTION OF THE INVENTION

The present invention will be explained in more detail hereinafter with reference to the embodiments shown in the drawings. It should be noted that the present invention is not limited thereto.

### Embodiment 1

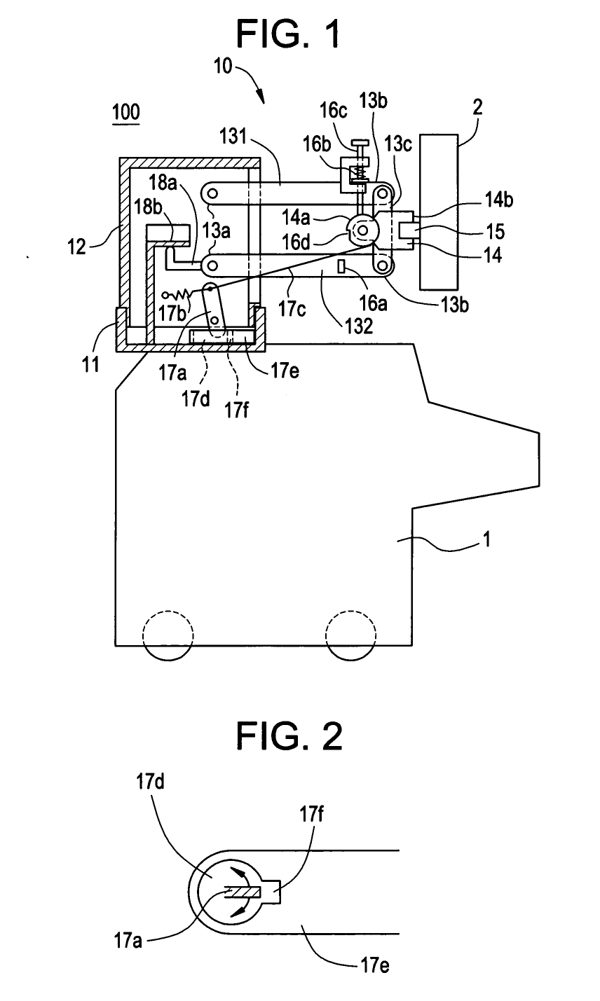

FIG. 1 is an explanatory view of a configuration of an ultrasound diagnostic apparatus 100 according to the embodiment 1. An essential part is overstated in the figure.

This ultrasound diagnostic apparatus 100 is provided with an ultrasound diagnostic apparatus body 1 that performs a control of a ultrasound probe or formation of an ultrasound image, an image display device 2 for displaying an ultrasound image, and an image display device supporting mechanism 10 that supports the image display device 2 to the ultrasound diagnostic apparatus body 1.

The image display device supporting mechanism 10 has a fixed section 11 fixedly mounted to the ultrasound diagnostic apparatus body 1, a rotary section 12 that is supported by the fixed section 11 and that can horizontally rotate with respect to the fixed section 11, arm members 131 and 132 that can pivot a second end 13b in the upward and downward directions with a first end 13a shaft-supported at the rotary section 12 as a support, a link member 13c that connects the second ends 13b of the arm members 131 and 132 for composing a parallel link mechanism, a neck member 14 that can pivot the other end 14b in a vertical plane with one end 14a shaft-supported by the link member 13c as a support, and a hinge member 15 whose other end is pivotable in the direction perpendicular to the pivoting direction of the neck member 14 with one end shaft-supported by the neck member 14 as a support and that supports the image display device 2 at the other end.

Accordingly, the image display device 2 is movable in the horizontal direction by the rotary section 12 and is movable in the upward and downward directions by the arm members 131 and 132, wherein the tilt angle of the display screen can be changed by the neck member 14 and the display screen can be swiveled by the hinge member 15. Since the arm members 131 and 132, form a parallel link mechanism, the display device 2 can be moved in the upward and downward directions without changing the tilt angle of the display screen.

A stopper 16a is mounted to the arm member 132. Further, held at the arm member 131 is a rod 16c that is urged downward by a spring 16b. One end 14a of the neck member 14 takes a cam shape and has a cutout 16d formed thereon.

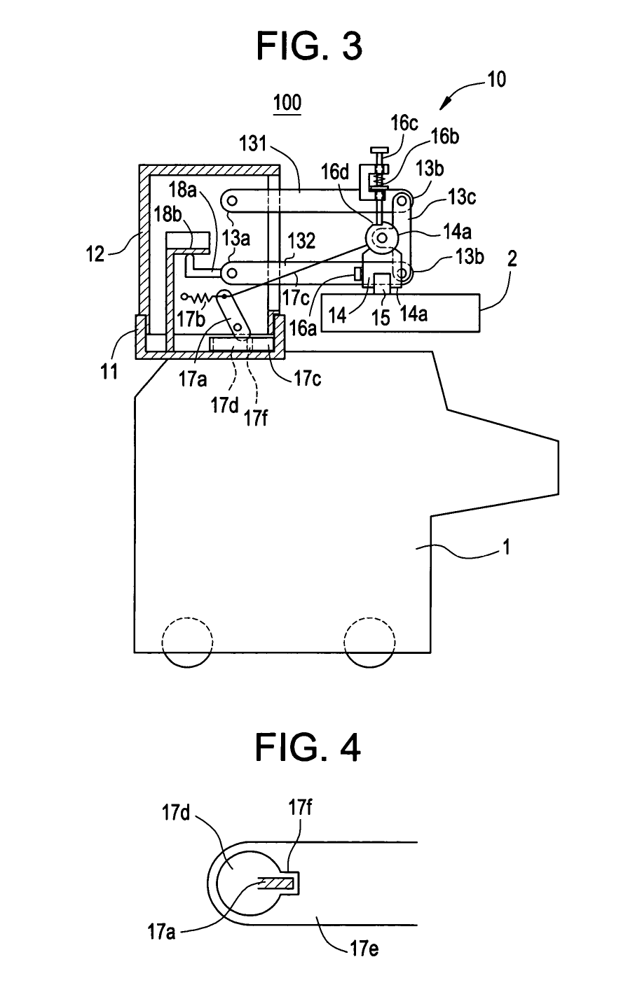

When the image display device 2 is thrown down such that the display screen faces downward, the stopper 16a is brought into contact with the neck member 14 as shown in FIG. 3. Further, the lower end of the rod 16c is inserted into the cutout 16d. This locks the tilt angle of the display screen with the state where the image display device 2 is thrown down with the display screen facing downward.

Returning to FIG. 1, a lever 17a is shaft-supported by the rotary section 12. The upper end of the lever 17a is pulled by a spring 17b, one end of which is fixed to the rotary section 12. Further, the upper end of the lever 17a and the one end 14a of the neck member 14 are connected by a wire 17c. Therefore, the angle of the lever 17a is determined according to the tilt angle of the image display device 2.

On the other hand, arranged at the fixed section 11 is a frame member 17e for forming an enclosing space 17d that encloses the surrounding of the lower end of the lever 17a. A cutout 17f is formed at a part of the enclosing space 17d.

With a state where the image display device 2 is not thrown down with the display screen facing downward, the surrounding of the lower end of the lever 17a is enclosed by the enclosing space 17d, but it is not inserted into the cutout 17f as shown in FIG. 2. Therefore, the rotary section 12 is rotatable.

On the other hand, when the image display device 2 is thrown down such that the display screen faces downward in the horizontal position in which the lower end of the lever 17a is inserted into the cutout 17f, the lower end of the lever 17a is inserted into the cutout 17f as shown in FIG. 4, so that the rotary section 12 cannot rotate. Thus, the horizontal position is locked.

In order to release the locking state shown in FIG. 3, an operator may pull up the rod 16c against the urging force of the spring b to stand up the image display device 2 such that the display screen faces frontward. This brings the apparatus into the state shown in FIG. 1, whereby the lock of the tilt angle is released and with this, the lock of the horizontal position is released.

The lower end of the lever 17a is not inserted into the cutout 17f even if the image display device 2 is thrown down so as to face the display screen downward, other than the horizontal position shown in FIG. 4, with the result that the image display device 2 cannot be thrown down until the tilt angle is locked. Therefore, the horizontal position is also not locked.

Returning to FIG. 1, a bar-like stopper 18a is projectingly mounted at the first end 13a of the arm member 132. On the other hand, a plate-like guide member 18b for regulating the position of the upper end of the stopper member 18a is mounted at the fixed section 11.

Even if the arm members 131 and 132 try to move in the downward direction from the height shown in FIG. 1 or FIG. 3 at the horizontal position where the arm members 131 and 132 face frontward, they cannot move in the downward direction since the stopper member 18a and the guide member 18b are brought into contact with each other. Thus, the interference between the ultrasound diagnostic apparatus body 1 and the image display device 2 can be prevented.

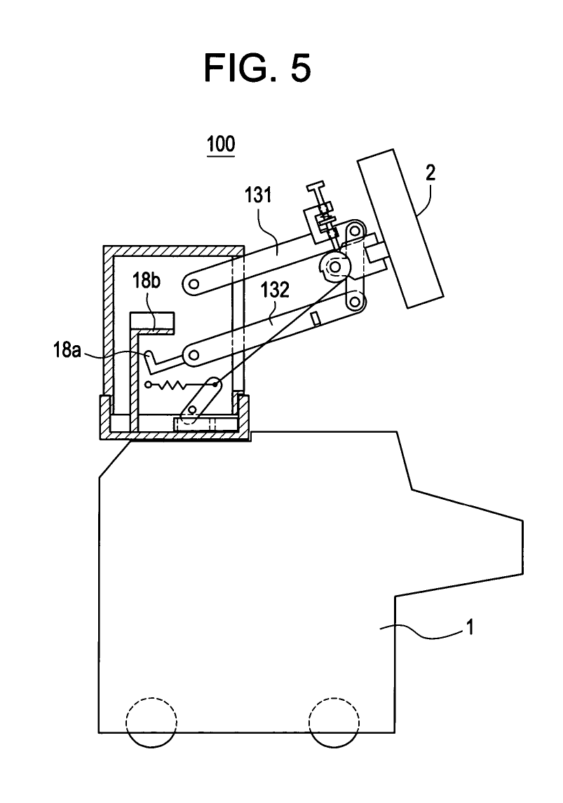

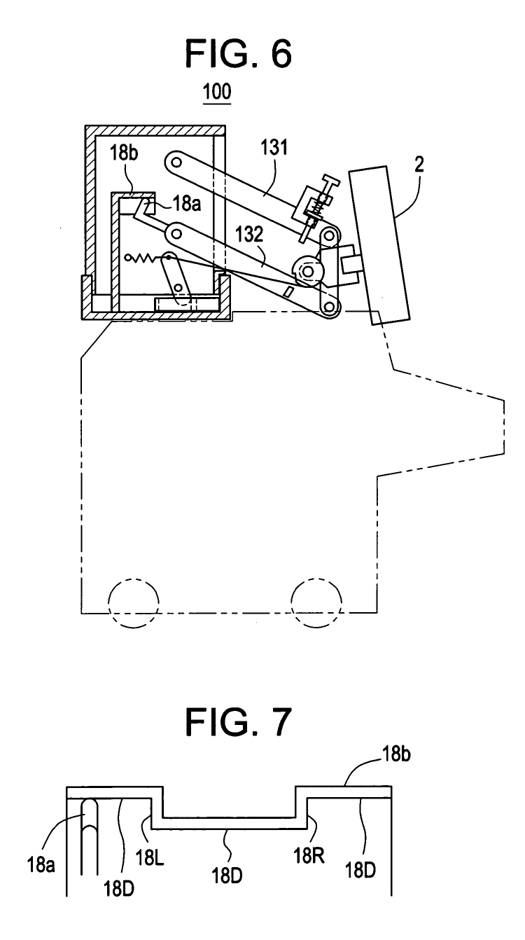

On the other hand, in case where the arm members 131 and 132 move in the upward direction from the height shown in FIG. 1 or FIG. 3, the stopper member 18a does not come in contact with the guide member 18b as shown in FIG. 5, whereby they can move in the upward direction. This is because, even if they move in the upward direction, the ultrasound diagnostic apparatus body 1 and the image display device 2 do not interfere with each other.

In case where the image display device 2 is located at the position higher than the height shown in FIG. 1 or FIG. 3, the horizontal movement is not restricted. This is because, even if the horizontal movement is allowed, the ultrasound diagnostic apparatus body 1 and the image display device 2 do not interfere with each other.

At the position where the rightmost point of the image display device 2 comes at the left side from the leftmost point of the ultrasound diagnostic apparatus body 1 or where the leftmost point of the image display device 2 comes at the right side from the rightmost point of the ultrasound diagnostic apparatus body 1, the height of the guide member 18b is higher than the height shown in FIG. 1 or FIG. 3, so that the arm members 131 and 132 can move in the downward direction from the height shown in FIG. 1 or FIG. 3. This is because, even if they move in the downward direction, the ultrasound diagnostic apparatus body 1 and the image display device 2 do not interfere with each other.

FIG. 7 is a front view showing an essential section of the stopper member 18a and the guide member 18b.

The upper end of the stopper member 18a is brought into contact with the lower face 18D of the guide member 18b, whereby the movement of the image display device 2 in the downward direction is restricted.

Further, the side face at the upper end of the stopper member 18a is brought into contact with the vertical faces 18L and 18R of the guide member 18b, whereby the movement of the image display device 2 in the horizontal direction is restricted.

Specifically,. at the vertical position where the rightmost point of the image display device 2 is positioned at the left side from the leftmost point of the ultrasound diagnostic apparatus body 1 and where the downmost point of the image display device 2 lowers from the topmost point of the ultrasound diagnostic apparatus body 1, the image display device 2 cannot horizontally move in the rightward direction, even if it is attempted to horizontally move the image display device 2 in the rightward direction such that the rightmost point of the image display device 2 is positioned at the right from the leftmost point of the ultrasound diagnostic apparatus body 1, since the side face at the upper end of the stopper member 18a and the right vertical face 18R of the guide member 18b are brought into contact with each other. Further, at the vertical position where the leftmost point of the image display device 2 is positioned at the right side from the rightmost point of the ultrasound diagnostic apparatus body 1 and where the downmost point of the image display device 2 lowers from the topmost point of the ultrasound diagnostic apparatus body 1, the image display device 2 cannot horizontally move in the leftward direction, even if it is attempted to horizontally move the image display device 2 in the leftward direction such that the leftmost point of the image display device 2 is positioned at the left from the rightmost point of the ultrasound diagnostic apparatus body 1, since the side face at the upper end of the stopper member 18a and the left vertical face 18L of the guide member 18b are brought into contact with each other. Thus, the interference between the ultrasound diagnostic apparatus body 1 and the image display device 2 can be prevented.

In summary, in order to avoid the interference between the ultrasound diagnostic apparatus body 1 and the image display device 2, the horizontally movable range and the vertically movable range may be restricted according to the horizontal position and the vertical position of the image display device 2 as described below.

(a) At the position where the downmost point of the image display device 2 is positioned above the topmost point of the ultrasound diagnostic apparatus body 1 and the rightmost point of the image display device 2 is positioned between the leftmost point and the rightmost point of the ultrasound diagnostic apparatus body 1, the vertically movable range is restricted such that the downmost point of the image display device 2 does not lower from the topmost point of the ultrasound diagnostic apparatus body 1.

(b) At the position where the downmost point of the image display device 2 is positioned above the topmost point of the ultrasound diagnostic apparatus body 1 and the leftmost point of the image display device 2 is positioned between the leftmost point and the rightmost point of the ultrasound diagnostic apparatus body 1, the vertically movable range is restricted such that the downmost point of the image display device 2 does not lower from the topmost point of the ultrasound diagnostic apparatus body 1.

(c) At the vertical position where the rightmost point of the image display device 2 is positioned at the left side from the leftmost point of the ultrasound diagnostic apparatus body 1 and the downmost point of the image display device 2 is positioned below the topmost point of the ultrasound diagnostic apparatus body 1, the horizontally movable range is restricted such that the rightmost point of the image display device 2 does not move to the right from the leftmost point of the ultrasound diagnostic apparatus body 1.

(d) At the vertical position where the leftmost point of the image display device 2 is positioned at the right side from the rightmost point of the ultrasound diagnostic apparatus body 1 and the downmost point of the image display device 2 is positioned below the topmost point of the ultrasound diagnostic apparatus body 1, the horizontally movable range is restricted such that the leftmost point of the image display device 2 does not move to the left from the rightmost point of the ultrasound diagnostic apparatus body 1.

The following effects can be obtained from the ultrasound diagnostic apparatus body 1 of the embodiment 1.

(1) The image display device 2 is supported by the ultrasound diagnostic apparatus body 1 via the image display device supporting mechanism 10 so as to be horizontally and vertically movable and so as to be capable of changing the tilt angle of the display screen of the image display device 2, thereby making it possible to move the display screen for the ultrasound image to the position and to have the posture by which the display screen is easy to be seen.

(2) The tilt angle of the display screen is locked and the horizontal movement is locked with the image display device 2 thrown down, when not used, whereby it is easy to carry.

(3) The horizontally movable range and the vertically movable range are restricted according to the horizontal position and the vertical position of the image display device 2, thereby being capable of avoiding the interference between the ultrasound diagnostic apparatus body 1 and the image display device 2.

### Embodiment 2

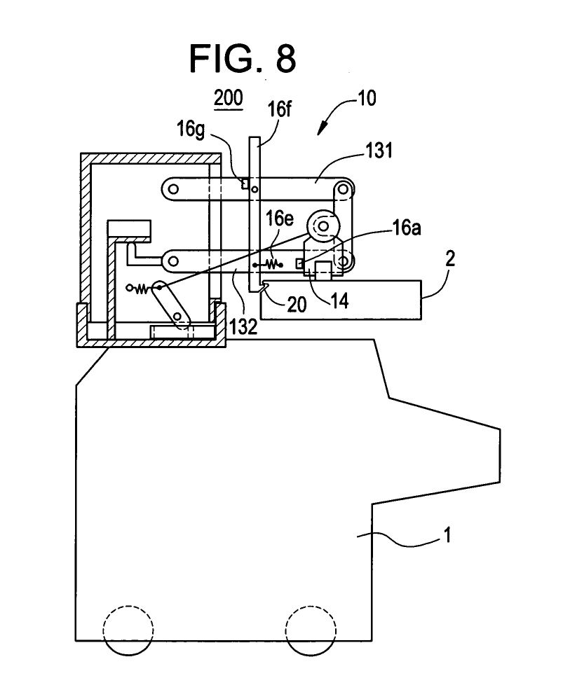

FIG. 8 is a construction explanatory view for showing an ultrasound diagnostic apparatus 200 according to the embodiment 2. It should be noted that an essential part is overstated in the figure.

This ultrasound diagnostic apparatus 200 has the configuration using a spring 16e, lever 16f, stopper 16g and cutout 20 instead of the spring 16b, rod 16c and cutout 16d.

The lever 16f is shaft-supported by the arm member 131 and urged by the spring 16e so as to rotate in the counterclockwise direction in FIG. 8. The stopper 16g restricts that the lever 16f rotates in the counterclockwise direction from the vertical angle.

The cutout 20 is formed at the lower end face of the image display device 2.

When the image display device 2 is thrown down with the display screen facing downward, the stopper 16a is brought into contact with the neck member 14 as shown in FIG. 8. Further, the lower end of the lever 16f is inserted into the cutout 20. This makes it possible to lock the tilt angle of the display screen with the state where the image display device 2 is thrown down with the display screen facing downward.

In order to release the locking state shown in FIG. 8, an operator may pull the lever 16f toward him/her against the urging force of the spring 16e, and then, he/she may raise the image display device 2 such that the display screen faces frontward.

### Embodiment 3

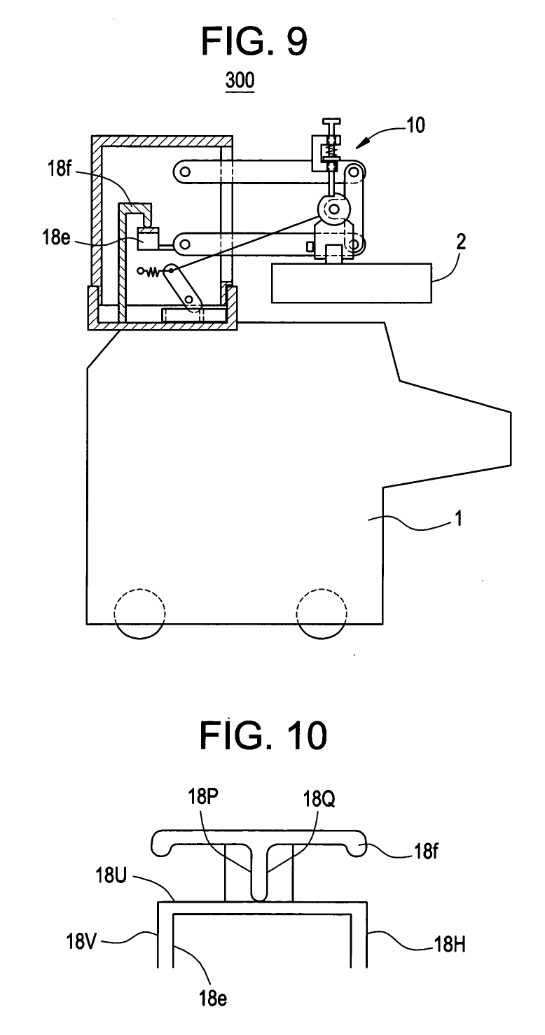

FIG. 9 is a construction explanatory view for showing an ultrasound diagnostic apparatus 300 according to the embodiment 3. It should be noted that an essential part is overstated in the figure.

This ultrasound diagnostic apparatus 300 has the configuration using a plate-like hook 18e and cam-shaped member 18f, instead of the stopper 18a and the guide member 18b of the ultrasound diagnostic apparatus 100 in the embodiment 1.

As shown in FIG. 10, the upper face 18U of the hook 18e is brought into contact with the lower end of the cam-shaped member 18f, whereby the downward movement of the image display device 2 is restricted. Further, the vertical face 18V of the hook 18e is brought into contact with the right face 18Q of the cam-shaped member 18f, whereby the leftward movement of the image display device 2 is restricted. Moreover, the vertical face 18H of the hook 18e is brought into contact with the left face 18P of the cam-shaped member 18f, whereby the rightward movement of the image display device 2 is restricted.

Many widely different embodiments of the invention may be configured without departing from the spirit and the scope of the present invention. It should be understood that the present invention is not limited to the specific embodiments described in the specification, except as defined in the appended claims.

1. An ultrasound diagnostic apparatus comprising:
an ultrasound diagnostic apparatus body performing a control of an ultrasound probe or a formation of an ultrasound image;
an image display device that displays the ultrasound image; and
an image display device supporting mechanism that supports the image display device to the ultrasound diagnostic apparatus body,
wherein the image display device supporting mechanism supports the image display device so as to be horizontally movable and vertically movable and so as to be capable of changing the tilt angle of a display screen of the image display device, as well as is capable of supporting the image display device in a state where the image display device is thrown down with the display screen facing downward, and the image display device supporting mechanism comprises tilt lock means for locking the tilt angle of the display screen in the state where the image display device is thrown down with the display screen facing downward, and movable range restricting means for restricting a horizontally movable range and a vertically movable range in accordance with the horizontal position and the vertical position of the image display device.

2. An ultrasound diagnostic apparatus comprising:
an ultrasound diagnostic apparatus body performing a control of an ultrasound probe or a formation of an ultrasound image;
an image display device that displays the ultrasound image; and
an image display device supporting mechanism that supports the image display device to the ultrasound diagnostic apparatus body,
wherein the image display device supporting mechanism supports the image display device so as to be horizontally movable and vertically movable and so as to be capable of changing the tilt angle of a display screen of the image display device, as well as is capable of supporting the image display device in a state where the image display device is thrown down with the display screen facing downward.

3. An ultrasound diagnostic apparatus according to claim 2, comprising tilt lock means for locking the tilt angle of the display screen in the state where the image display device is thrown down with the display screen facing downward.

4. An ultrasound diagnostic apparatus according to claim 2, wherein the image display device supporting mechanism has horizontal movement lock means for locking the horizontal position of the image display device when the tilt angle is locked by the tilt lock means.

5. An ultrasound diagnostic apparatus according to claim 4, wherein the horizontal movement lock means returns the image display device to the horizontal position when the lock of the tilt angle by the tilt lock means is released and the image display device is raised such that the display screen faces frontward.

6. An ultrasound diagnostic apparatus according to claim 2, wherein the lock of the tilt angle by the tilt lock means is possible only in the state where the image display device is at a specific horizontal position.

7. An ultrasound diagnostic apparatus according to claim 2, wherein the image display device supporting mechanism has a rotary section that is rotatable in the horizontal direction with respect to the ultrasound diagnostic apparatus body, an arm section having a first end side supported by the rotary section and a second end side that can be pivoted in the upward and downward directions with the first end side as a support, and a neck section having one end supported by the second end side of the arm section and the other end side that can be pivoted in the upward and downward directions with the one end side as a support, the image display device being supported to the other end side.

8. An ultrasound diagnostic apparatus according to claim 7, wherein the tilt lock means is means for locking the pivot movement of the neck section.

9. An ultrasound diagnostic apparatus according to claim 7, wherein the horizontal movement lock means is means for locking the rotation of the rotary section.

10. An ultrasound diagnostic apparatus comprising:
an ultrasound diagnostic apparatus body performing a control of an ultrasound probe or a formation of an ultrasound image;
an image display device that displays the ultrasound image; and
an image display device supporting mechanism that supports the image display device to the ultrasound diagnostic apparatus body,
wherein the image display device supporting mechanism supports the image display device so as to be horizontally movable and vertically movable and has movable range restricting means for restricting a horizontally movable range and vertically movable range in accordance with the horizontal position and the vertical position of the image display device.

11. An ultrasound diagnostic apparatus according to claim 1, wherein the movable range restricting means restricts the vertically movable range such that, at the position where the downmost point of the image display device is positioned above the topmost point of the ultrasound diagnostic apparatus body and the rightmost point of the image display device is positioned between the leftmost point and the rightmost point of the ultrasound diagnostic apparatus body, the downmost point of the image display device does not lower from the topmost point of the ultrasound diagnostic apparatus body, the movable range restricting means restricts the vertically movable range such that, at the position where the downmost point of the image display device is positioned above the topmost point of the ultrasound diagnostic apparatus body and the leftmost point of the image display device is positioned between the leftmost point and the rightmost point of the ultrasound diagnostic apparatus body, the downmost point of the image display device does not lower from the topmost point of the ultrasound diagnostic apparatus body, the movable range restricting means restricts the horizontally movable range such that, at the vertical position where the rightmost point of the image display device is positioned at the left side from the leftmost point of the ultrasound diagnostic apparatus body and the downmost point of the image display device is below the topmost point of the ultrasound diagnostic apparatus body, the rightmost point of the image display device does not move to the right from the leftmost point of the ultrasound diagnostic apparatus body, and the movable range restricting means restricts the horizontally movable range such that, at the vertical position where the leftmost point of the image display device is positioned at the right side from the rightmost point of the ultrasound diagnostic apparatus body and the downmost point of the image display device is below the topmost point of the ultrasound diagnostic apparatus body, the leftmost point of the image display device does not move to the left from the rightmost point of the ultrasound diagnostic apparatus body.

12. An ultrasound diagnostic apparatus according to claim 10, wherein the image display device supporting mechanism includes a rotary section that is rotatable in the horizontal direction with respect to the ultrasound diagnostic apparatus body, and an arm section that has the first end side supported to the rotary section, and has the second end side that supports the image display device and can be pivoted in the upward and downward directions with the first end side as a support, the image display device being supported to the second end side.

13. An ultrasound diagnostic apparatus according to claim 12, comprising a neck section having one end supported by the second end side of the arm section and the other end side that can be pivoted in the upward and downward directions with the one end side as a support, the image display device being supported to the other end side.

14. An ultrasound diagnostic apparatus according to claim 12, wherein the movable range restricting means comprises a guide member that is located at a fixed position with respect to the ultrasound diagnostic apparatus body, and a stopper member that moves with the arm member, wherein the stopper member is brought into contact with a part of the guide member, thereby restricting the movable ranges of the rotary section and the arm section.

15. An ultrasound diagnostic apparatus according to claim 2, wherein the image display device supporting mechanism maintains the tilt angle of the display screen of the image display device fixed even when the vertical position of the image display device is changed.

16. An ultrasound diagnostic apparatus according to claim 2, wherein the image display device is a flat panel type image display device.

17. An ultrasound diagnostic apparatus according to claim 16, wherein the flat panel type image display device is a liquid crystal display device.

18. An ultrasound diagnostic apparatus according to claim 10, wherein the movable range restricting means restricts the vertically movable range such that, at the position where the downmost point of the image display device is positioned above the topmost point of the ultrasound diagnostic apparatus body and the rightmost point of the image display device is positioned between the leftmost point and the rightmost point of the ultrasound diagnostic apparatus body, the downmost point of the image display device does not lower from the topmost point of the ultrasound diagnostic apparatus body, the movable range restricting means restricts the vertically movable range such that, at the position where the downmost point of the image display device is positioned above the topmost point of the ultrasound diagnostic apparatus body and the leftmost point of the image display device is positioned between the leftmost point and the rightmost point of the ultrasound diagnostic apparatus body, the downmost point of the image display device does not lower from the topmost point of the ultrasound diagnostic apparatus body, the movable range restricting means restricts the horizontally movable range such that, at the vertical position where the rightmost point of the image display device is positioned at the left side from the leftmost point of the ultrasound diagnostic apparatus body and the downmost point of the image display device is below the topmost point of the ultrasound diagnostic apparatus body, the rightmost point of the image display device does not move to the right from the leftmost point of the ultrasound diagnostic apparatus body, and the movable range restricting means restricts the horizontally movable range such that, at the vertical position where the leftmost point of the image display device is positioned at the right side from the rightmost point of the ultrasound diagnostic apparatus body and the downmost point of the image display device is below the topmost point of the ultrasound diagnostic apparatus body, the leftmost point of the image display device does not move to the left from the rightmost point of the ultrasound diagnostic apparatus body.

19. An ultrasound diagnostic apparatus according to claim 10, wherein the image display device supporting mechanism maintains the tilt angle of the display screen of the image display device fixed even when the vertical position of the image display device is changed.

20. An ultrasound diagnostic apparatus according to claim 10, wherein the image display device is a flat panel type image display device.

21. An ultrasound diagnostic apparatus according to claim 20, wherein the flat panel type image display device is a liquid crystal display device.
